(19) United States
(12) Patent Application Publication
(10) Pub. No.: US 2008/0058705 A1
(43) Pub. Date: Mar. 6, 2008

(54) Arrangement for Therapy of Tumours

(75) Inventors: Hans Wiksell, Taby (SE); Gert Auer, Solna (SE); Vilhelm Ekstrand, Nacka (SE); Peter Harge, Saltsjobaden (SE)

Correspondence Address: YOUNG & THOMPSON, 745 SOUTH 23RD STREET, 2ND FLOOR, ARLINGTON, VA, 22202, US

(73) Assignee: VIBRATECH AB, Stockholm (SE)

(21) Appl. No.: 11/663,733

(22) PCT Filed: Sep. 22, 2005
(86) PCT No.: PCT/SE05/01395

§ 371 Date: May 22, 2007

Related U.S. Application Data
(60) Provisional application No. 60/612,788, filed on Sep. 27, 2004.

(30) Foreign Application Priority Data
Sep. 27, 2004 (EP) 04022985.8

(51) Int. Cl. A61N 1/02

(52) U.S. Cl. 604/21; 606/41; 607/3

(57) ABSTRACT

Arrangement for treatment of a tumour localized inside a body, comprising a needle that is intended to be inserted into said tumour, a ground electrode that is intended to be applied to the outer surface of the body, and a radio frequency generating device that is intended to apply radio frequency energy between the needle and the ground electrode such that heat is generated in tissue surrounding the needle. The arrangement further comprises novel temperature measurement means and means that provides for a better penetration of the needle into the tumour in comparison with prior art RFA arrangements.

### TECHNICAL FIELD

The present invention relates to an arrangement for the therapy of tumours, which is applicable to the Radio Frequency Ablation (RFA) technique. In comparison with prior art RFA arrangements, the arrangement according to the present invention comprises means that provides for a better penetration of the needle into the tumour. In a preferred embodiment of the present invention, the arrangement comprises temperature control means that provides for a more accurate temperature control. The inventive arrangement also provides for a more effective treatment of the tumour. The present invention also relates to a method for treating tumours which brings about the above mentioned advantages.

### BACKGROUND ART

Many women in the western world face the prospect of breast cancer. The tendency in modern breast cancer treatment has been towards less invasive local treatment of the tumour and it is also becoming more important in modern therapy to minimise the alteration of the breast configuration due to treatment. Breast conservation surgery has thus in many cases gained in favour over radical mastectomy. The goal of radical surgery is to remove all of the malignant breast tissue, often combined with lymph node resection, resulting in a considerable hospitalisation period and later need of breast restitution. Furthermore, a tumour free zone is resected to prevent local reoccurrence. With modern diagnostic methods, as routine mammography screening, many small tumours are detected. In cases where the tumour is still not spread, i.e. the majority of the small tumours being ≦10 mm, conventional breast surgery might be seen as an “overkill” procedure. Thus, minimally invasive approaches other than traditional surgery have been explored, with which other potential benefits often are obtained, such as reduced morbidity rates, reduced treatment duration, ability to perform therapy even when patients are in bad medical condition and possibility to keep the procedure on an outpatient basis.

One such approach is the Radio Frequency Ablation (RFA) technique, which causes thermal destruction of the tumour by coagulation necrosis. Liver tumours are the most common type of tumours that are treated with RFA but the treatment is now being performed in other areas of the body, such as the lung, bones, prostate and adrenal glands. The use of RFA in breast cancer treatment is relatively new but studies performed by the inventors have shown that the technique is well suited for treatment of breast tumours and especially ductal breast tumours.

The RFA monopolar technique involves inserting a needle directly into the tumour to be treated and applying a larger planar ground electrode to an outer surface of the body. When radio frequency (RF) energy, generated by a power source, is applied between the ground electrode and the needle, the former will act as a counter electrode and a current path is established between the electrodes. The current density at the needle will be much higher than at the ground electrode, thus the latter is often referred to as the indifferent electrode and associated with no temperature elevation. Temperature elevation in the tissue is caused by ion agitation, which is converted by means of friction into heat, thus the RF energy heats the tissue by way of the tissue's electric resistance. The heat, that is generated in the tissue surrounding the needle, destroys the tumour cells, without nerve stimulation and pain. The denaturised cells are left in-situ and are later resorbed by the body. In order to avoid regrowth of the tumour, a layer of surrounding tissue, a safety zone, often needs to be destroyed as well. The total volume affected by the excess temperature is known as the lesion volume and thus the shape and size of the affected area is known as the lesion shape and lesion size, respectively.

It is well known that tissue exposed to excess temperature during a sufficient long time is completely killed. In a technique generally referred to as hyperthermia, the temperature generated is approximately 42-43° C. With an excess temperature in this range, the exposure time needed to effectively treat a tumour, is normally a number of minutes, sometimes up to one hour, depending on for instance the lesion size. Thus, hyperthermia temperatures within the field of RFA inevitably prolongs the treatment time of the patient, brings about unnecessary suffering and often produces non-uniform lesioning of the tumour.

The tissue heating is constantly cooled during treatment, both passively through thermal conduction as well as actively through blood perfusion.

Thus, the latter contributes to a large extent to remove heat. However, blood perfusion is affected by heat. Moderate increases in temperature also increase blood perfusion, but at higher excess temperatures, the blood perfusion collapses. Thus the latter results in that the temperature diffusion increases enormously.

Moreover, the temperature of the treated tissue is a parameter of great importance. For instance, at sufficiently high temperatures, tissue adjacent to the electrode surface will get charred and thus impair further heating of the tissue, due to the drastic decrease in conductivity. In certain applications it may also be important to avoid that gas bubbles are created at the needle surface which tend to isolate the electrode from the tissue. The size of the lesion volume is limited because of the above mentioned effects.

Therefore, in order to obtain an effective treatment of the tumour, it is of importance that the temperature is monitored and controlled effectively and that the needle is cooled in an effective way.

The output-power of the RF source regulates the generated temperature in the tissue. Within the RFA technique, it is known to measure for instance the impedance in the circuit comprising the needle and the indifferent ground electrode as well as measuring the temperature at the needle-tip during treatment and adjusting the voltage between the electrodes according to the measured values of the impedance and the temperature. It is also known to continuously feed the values of the impedance and the temperature during treatment to a control unit, which will send feed-back signals to the RF-source, increasing or decreasing the output-power of said source.

An increase in the impedance indicates that for instance bubbles have been created or that tissue charring has occurred. In order to cool the needle, to prevent tissue charring and bubble formation, it is known to use needles which utilize cooling media in the interior of the needle. One of the results of having such cooling media inside the needle, is that the true maximum temperature of the tissue temperature, due to temperature deviation, is not located at the needle surface but at a certain distance from said needle inside the tissue. This makes it difficult to measure the true maximum temperature of the tissue and the known means for temperature control of the tissue temperature have not proven to be satisfying enough.

In the recent years, it has also been possible to detect smaller and smaller tumours, for instance through the technical development of screening mammography and ultrasound diagnostic scanning. It is not unusual to detect tumours which are in the size of just a few millimetres. Especially these small tumours but also tumours of sizes 10-20 mm in diameter are frequently non-palpable and undergo anatomical distortion or at least dislocation when hit by the needle. The reason is that the glandular breast tissue is surrounded by a loose fatty and connective tissue, which makes it difficult to correctly position and move the needle into the tumour and the problem aggravates if the tumour is hard or fibrous, which is most often the case. Thus, the insertion of needles into the tumour is often difficult or even impossible.

At the present the RFA technique is, when it is applied in cancer treatment, most often viewed as palliative, but not curative and the technique is in most cases used to shrink tumours so that surgery can be avoided. Thus, there is a need for improvements of the RFA technique, such as means that provides for a more effective penetration of the tumour to be treated, especially small and hard fibrous tumours. There is also a need for a shortening of the actual treatment time and a more effective killing of the tumour cells. Also, there is a need for a more accurate temperature control of the tissue temperature.

### DISCLOSURE OF THE INVENTION

Thus, the object of the invention is to provide an arrangement for the treatment of tumours with the RFA technique, which arrangement provides means for a more effective penetration of the tumour to be treated in comparison with prior art RFA arrangements.

This object is achieved by an arrangement according to the preambles of the independent claim and provided by the features according to the characteristic portion of the independent claim.

In a preferred embodiment, the arrangement according to the present invention provides means for a more accurate temperature control of the tissue temperature during treatment in comparison with prior art RFA arrangements. Said arrangement also treats the tumour in a more effective way in comparison with prior art RFA arrangements.

The invention also relates to a method of using the inventive arrangement. Preferred embodiments are set forth in the dependent claims.

### BRIEF DESCRIPTION OF DRAWINGS

FIG. 1 schematically shows the inventive arrangement with a first set of temperature measurement means,

FIG. 2 schematically shows the inventive arrangement with a second set of temperature measurement means,

FIG. 3 schematically shows a cross section of the needle of the inventive arrangement,

FIG. 4 shows the flow dependent measured temperatures at different calibration temperatures.

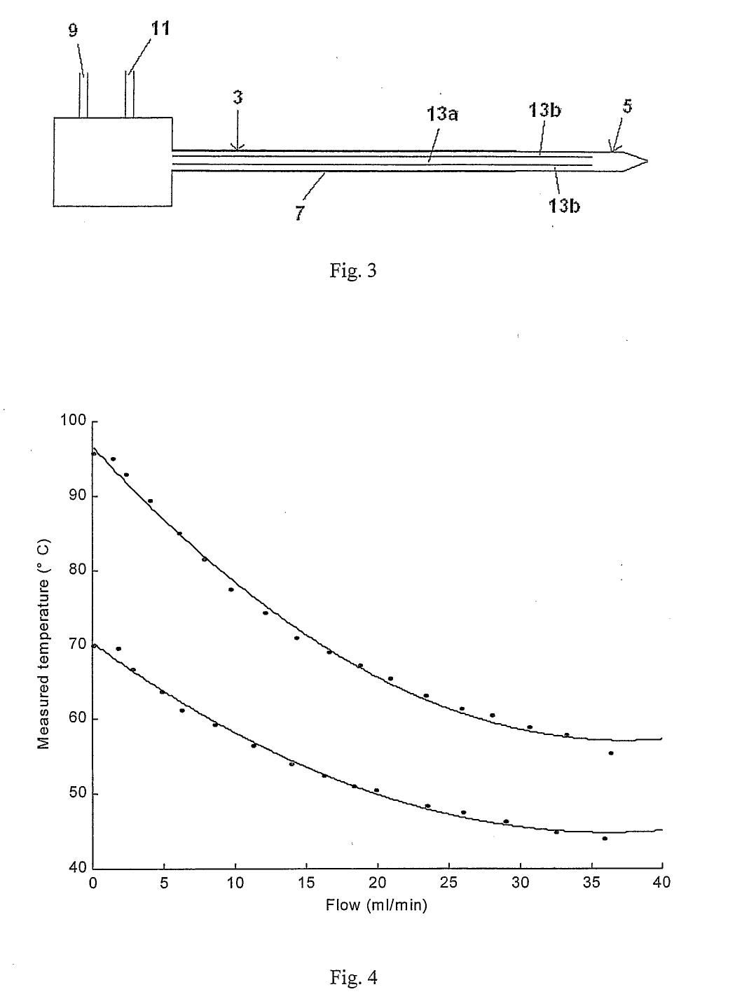

The inventive arrangement comprises a needle 2 (FIG. 3) and a planar ground electrode 4, that is to be applied to an outer surface of the patient's body, preferably the back of the patient. The dimensions of the needle 2 depend on for instance the size and shape of the tumour 1 to be treated. Preferably the needle 2 has an active part of a length of approximately 5-100 mm and a diameter in the range of 1-4 mm. The arrangement further comprises a low impedance RF generating device 10, that is adapted to apply RF energy between the ground electrode 4 and the active part of the needle 2, when the needle 2 is correctly positioned inside the tumour 1 to be treated. When RF energy is applied, the needle 2 acts as an active electrode, such that a current path is established between the electrodes 2, 4, generating heat in a spherical lesion 12 of tissue surrounding the needle 2, as described above.

According to the invention, when for instance a breast tumour is treated, the tissue temperature can be considerably higher than the commonly used hyperthermia temperature. Said tissue temperature can for instance be in the range of 50-99° C., i.e. the so called thermotherapy temperature. Due to the dissimilar physical parameters of tumour and adipose (fat) tissue, the heat “is drawn” to the tumour. The tumour has a higher electric conductivity than the surrounding adipose tissue. As a consequence, the tumour attracts the electric energy from the fat. Thus, the specific absorption rate (SAR) is relatively higher in the tumour than in the adipose tissue. In fact, the inventors have observed that thin tumour strips extending from the core tumour are killed but the surrounding fatty tissue is unaffected. In order to achieve an effective treatment, the time needed to maintain the elevated temperature, is dependent on the lesion size and shape. Normally this time is in the range of 5-15 minutes, thus a shorter treatment time than would be needed with a temperature within the hyperthermia temperature range, in the treatment of the same tumour(s). It is however to be understood that when other types of tumours are treated, such as for instance tumours in the liver, the treatment temperature could be even higher, for instance up to approximately 130° C.

In any case, the output power of the RF generating device regulates the elevated tissue temperature. Generally, the RF generating device operates in the range of 5-100 W, preferably 20-60 W, depending on for instance the breast configuration when a breast tumour is treated, the used needle diameter and changes in tissue properties during the treatment. The applied RF frequency may be in the range of 0.2-20 MHz but is preferably approximately 0.5-1.5 MHz. According to the invention the power of the RF generating device is automatically adjusted during the treatment as described below.

With reference to FIG. 3, the needle 2 of the inventive arrangement is provided with an insulated part 3 and a non-insulated part 5, by covering the exterior of the needle with an insulating material 7, for instance Teflon®. The insulated part of the needle can be slideable over the needle to enable adjustment of the active electrode length.

According to a preferred embodiment of the inventive arrangement, the needle 2 that is to be inserted to the tumour 1 is further provided with means that enables circulation of a cooling liquid inside the needle 2. The cooling liquid is guided into the needle through a liquid inlet 9 and along a liquid inflow channel 13a, provided in the middle of the needle 2 along the horizontal axis of said needle 2. At the needle tip the cooling liquid changes flow direction and is guided back through a liquid outflow channel 13b, i.e. the empty space between the liquid inflow channel 13a and the outer surface of the needle, and out through a liquid outlet 11. Liquid inlet 9 and liquid outlet 11 are preferably provided with Luer-Lock connections (not shown). The means that enables circulation of a cooling liquid inside the needle 2, further comprises a liquid flow generating device 14, for instance a pump or the like, which is adapted to direct the liquid into the needle 2 through liquid inlet 9, through inflow and outflow channels 13a,b and out through liquid outlet 11, preferably with a constant, pre-determined flow rate. The diameter of the channels 13a,b is thus preferably sufficient to permit an equal flow in the liquid inflow and outflow channels. The preferred flow rate is in the range of 5-30 ml/min.

The cooling liquid is preferably a sterile isotone liquid in order to protect the patient against possible leakage.

The needle 2 is preferably provided as a disposable article. Alternatively, the needle 2 is adapted to be sterilized, preferably by the use of autoclavation. However other conventionally used sterilization methods may be used as well.

According to a preferred embodiment the arrangement is further provided with temperature control means. Said means comprises means (not shown) adapted to measure the flow value, Q, of the liquid circulating inside the needle 2, preferably provided in the liquid flow generating device 14. Said means is for instance a generally known flow measuring sensor or the like.

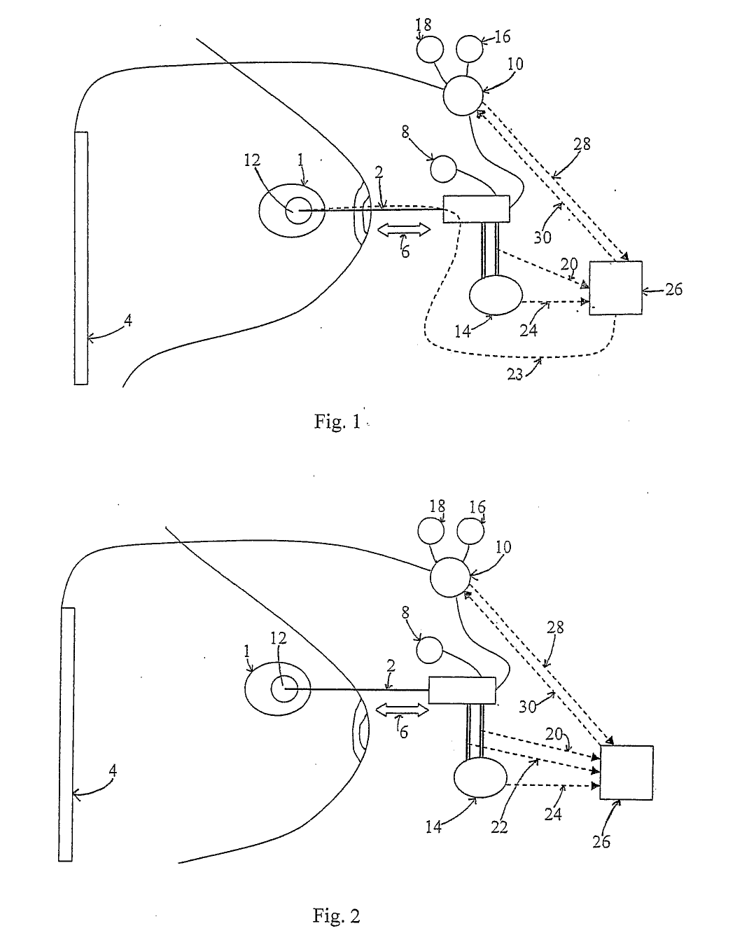

The temperature control means further comprises temperature measurement means that in a first embodiment comprises (a) generally known thermosensor(s) or optical fibres (not shown) incorporated in the non-insulated part of the needle 2 as well as a thermosensor adapted to measure the temperature of the inlet liquid, T₁, preferably provided in the liquid inlet 9 (first set of temperature measurement means, FIG. 1). In this instance, the temperature of the needle, Tₘₑₐₛₑ, and T₁, as measured by the first set of temperature measurement means, as well as the value of the liquid flow, Q=dV/dt (ml/s), is continuously fed 23, 20, 24 to a control unit 26, preferably a computer or the like. The control unit 26 is provided with means that according to the value of Tₘₑₐₛₑ, T₁ and the value of the flow Q, will calculate the true temperature of the tissue, Tₜᵢₛₛᵤₑ, as explained in further detail below.

Alternatively, the temperature measurement means may in a second embodiment comprise means (not shown) for measurement of the temperature value of the inlet liquid, T₁, as well as the temperature value of the outlet liquid, T₂ (second set of temperature measurement means, FIG. 2). Said means is preferably generally known thermosensors, provided for instance in the liquid inlet 9 and the liquid outlet 11, respectively. In the second embodiment (FIG. 2), T₁, T₂ and the value of the liquid flow, Q=dV/dt (ml/s), is continuously fed 20, 22, 24 to the control unit 26. In this instance, the control unit 26 is provided with means that according to the value of the temperature difference between T₁ and T₂, i.e. T_{diff}=T₂−T₁, and the value of the flow Q, will calculate the true temperature of the tissue, Tₜᵢₛₛᵤₑ.

According to the preferred embodiment of the inventive arrangement, the control unit 26 is also continuously fed 28 with values of the impedance, Z, in the circuit comprising the needle 2 and the indifferent ground electrode 4, as well as the output power, P=U*I, of the RF generating device 10 that regulates the generated elevated tissue temperature. Thus, the inventive arrangement comprises generally known means 16 adapted to measure the impedance and generally known means 18 adapted to measure the output power. The control unit 26 is accordingly further provided with means that depending on the values of Z, P and the true tissue temperature, Tₜᵢₛₛᵤₑ, will send feed-back signals 30 to the RF generating device 10, increasing or decreasing the output-power of said RF generating device, depending on if Tₜᵢₛₛᵤₑ is to be increased or decreased, respectively.

The power needed to maintain a certain tissue temperature, also referred to as the heat resistance, is a good indicator of the perfusion status of the tissue, i.e. the degree of tissue destruction, and the perfusion collapse might be a good indicator on the quality of the therapy session. Furthermore the measured impedance between the treatment and the indifferent electrodes is a good indicator of the status around the electrode, i.e. if charring and/or bubbles is/are present. Thus the control unit 26 is preferably provided with means to calculate the heat resistance as a tool to monitor the tissue perfusion.

The use of the value of Q when determining the true tissue temperature value, Tₜᵢₛₛᵤₑ, as well as using said values for sending feed-back signals to the RF generating device provides for a more accurate temperature control of the tissue temperature in comparison with prior art RFA arrangements.

The inventive arrangement functions in combination with either of above mentioned means for temperature measurement. The use of the first set of temperature measurement means, i.e. for instance thermosensor(s) incorporated in the needle is a straight forward temperature measurement method. The measurement of T₁, T₂, respectively, may however provide for a less expensive temperature measurement than the use of thermosensor(s) in the needle, since if the dimensions of the needle are decreased, the incorporation of thermosensors in the needle may be problematic and a rather expensive procedure.

The low level signal obtained from the thermosensors may suffer from interference from the applied RF-power field. In this instance the arrangement is preferably provided with low-pass filtering means (not shown) in order to filter the RF-signal to obtain high quality measurements in the extreme RF-field environment. Moreover, the arrangement is preferably designed as cardiac floating (CF) to guarantee patient leakage current (PLC) and earth leakage current (ELC), according to EN 60-601-1 international standard and EC.

According to the present invention, the arrangement is provided with a longitudinal movement means 8, adapted to apply an oscillating longitudinal movement 6 to the needle 2. Longitudinal oscillation 6 of the needle 2 with suitable frequency and amplitude will due to it's acceleration component effectively penetrate even small and hard tumours, for instance a tumour of just a few millimeteres, and the inventors have in fact discovered that the resistance at penetration may be reduced by as much up to ten times when a frequency of 250 Hz is applied. Depending on for instance the dimensions of the needle 2 and the size, condition and localization of the tumour 1, the operator of the arrangement, for instance the physician responsible for the treatment, is capable of, and free to, choose a suitable frequency, amplitude and wave form that is to be applied to the needle 2 when said needle is about to penetrate the tumour. Preferably the applied sinusoidal frequency is in the range of 30-300 Hz and the amplitude in the range of 0-4 mm. The penetration force is decreasing with increasing frequency and amplitude and the acceleration forces increase with the parameters.

The oscillating movement 6 of the needle 2 is applied in order to provide for a better penetration of the needle into the tumour 1.

Calculation of the true tissue temperature using the first set of temperature measurement means

The true tissue temperature Tₜᵢₛₛᵤₑ, is determined by using the values of Tₘₑₐₛ and the value of the flow Q, for instance according to equation (1):
dT=Tₜᵢₛₛᵤₑ−Tₘₑₐₛ=k₁(Tₜᵢₛₛᵤₑ)*Q+k₂(Tₜᵢₛₛᵤₑ)*Q² (1)
wherein dT is the temperature gradient over the needle and k₁ and k₂ are constants.

k₁ and k₂ are found using calibration. For instance, FIG. 4 shows the flow dependent measured temperatures, Tₘₑₐₛₑ, at different calibration temperatures (serving as fictitious tissue temperatures), using a bath unit kept at temperatures 70° C. (lower curve) and 95° C. (upper curve). In the calibration measurements the bath unit was kept at said calibration temperatures, respectively, and Tₘₑₐₛ was measured at 20 different flow rates. The cooling liquid circulating inside the needle was kept at 15° C. Using least square algorithms, k₁ and k₂ were calculated for tissue temperatures 70 and 95° C. At a constant flow rate there is a linear relationship between Tₘₑₐₛ and Tₜᵢₛₛᵤₑ. Thus Tₜᵢₛₛᵤₑ is calculated by linear extrapolation between the 70 and 95° C. curves in all flows. If the temperature of the inlet flow is changed the constants k₁ and k₂ are also changed according to a known relation.

Calculation of the true tissue temperature using the second set of temperature measurement means

The true tissue temperature Tₜᵢₛₛᵤₑ is in this scheme determined by the difference in inlet and outlet temperatures T_{diff}=T₁−T₂ instead of Tₘₑₐₛ. Otherwise, the procedure preferably follows the same scheme as the above described measurement calculation method.

Even though the inventive arrangement has been described by means of a procedure for treatment of a human breast tumour, the arrangement shall not be regarded as being limited to such. The arrangement is fully functional with other kinds of tumours, such as prostate tumours, liver tumours, as well as tumours in animals.

It will be understood that the invention is not restricted to the above-described exemplifying embodiments thereof and that several conceivable modifications of the invention are possible within the scope of the following claims.

1. Arrangement for treatment of a tumour localized inside a body, comprising a needle (2) that is intended to be inserted into said tumour, a ground electrode (4) that is intended to be applied to the outer surface of the body, and a radio frequency generating device (10) that is intended to apply radio frequency energy between the needle and the ground electrode such that heat is generated in tissue surrounding the needle characterized in that the arrangement comprises longitudinal movement means (8) adapted to apply an oscillating longitudinal movement to the needle, preferably applied upon insertion of the needle into said tumour.

2. Arrangement according to claim 1 characterized in that the needle (2) is provided with a liquid inlet (9) communicating with a liquid inflow channel (13a) and a liquid outlet (11) communicating with a liquid outflow channel (13b), said channels are provided in the interior of the needle (2) along the horizontal axis of said needle; and in that the arrangement is provided with a liquid flow generating device (14) adapted to generate a flow of liquid in said channels (13a, 13b).

3. Arrangement according to claim 2 characterized in that the arrangement is provided with means adapted to measure the flow value of the liquid.

4. Arrangement according to claim 2 characterized in that the arrangement comprises means adapted to measure the temperature of the tissue, comprising thermosensor(s) incorporated in the needle (2), means adapted to measure the temperature of the liquid in the liquid inlet (9) and said means adapted to measure the flow value of the liquid.

5. Arrangement according to claim 2 characterized in that the arrangement comprises means adapted to measure the temperature of the tissue, comprising means adapted to measure the temperature of the liquid in the liquid inlet (9), means adapted to measure the temperature of the liquid in the liquid outlet (11) and said means adapted to measure the flow value of the liquid.

6. Arrangement according to claim 1 characterized in that the arrangement comprises means (16) for measuring the impedance in the circuit comprising the needle (2) and the ground electrode (4) and means (18) adapted to measure the output power of the radio frequency generating device (10).

7. Arrangement according to claim 2 characterized in that the arrangement comprises a control unit (26), said control unit is: adapted to receive values of:
a) the liquid flow in the channels (13a, 13b) of the needle (2),
b) the temperature as measured by the thermosensor(s) incorporated in the needle (2), and the temperature of the liquid in the liquid inlet (9),
c) the impedance in the circuit comprising the needle (2) and the ground electrode (4),
d) the output power of said radio frequency generating device (10) provided with means adapted to calculate the tissue temperature depending on the values of a) and b), and adapted to send signals (30) to the radio frequency generating device (10) with instructions to decrease or increase the output power of said radio frequency generating device depending on if the tissue temperature is intended to be decreased or increased.

8. Arrangement according to claim 2 characterized in that the arrangement comprises a control unit (26), said control unit is: adapted to receive values of:
a) the liquid flow in the channels (13a, 13b) of the needle (2) and
b) the temperature of the liquid in the liquid inlet (9) and the temperature of the liquid in the liquid outlet (11);
c) the impedance in the circuit comprising the needle (2) and the ground electrode (4),
d) the output power of said radio frequency generating device (10) provided with means adapted to calculate the tissue temperature depending on the values of a) and b), and adapted to send signals (30) to the radio frequency generating device (10) with instructions to decrease or increase the output power of said radio frequency generating device depending on if the tissue temperature is intended to be decreased or increased.

9. Arrangement according to claim 7 characterized in that the control unit (26) is provided with means to calculate the power needed of the radio frequency generating device (10) in order to maintain a predetermined tissue temperature.

10. (canceled)

11. Arrangement according to claim 3 characterized in that the arrangement comprises means adapted to measure the temperature of the tissue, comprising thermosensor(s) incorporated in the needle (2), means adapted to measure the temperature of the liquid in the liquid inlet (9) and said means adapted to measure the flow value of the liquid.

12. Arrangement according to claim 3 characterized in that the arrangement comprises means adapted to measure the temperature of the tissue, comprising means adapted to measure the temperature of the liquid in the liquid inlet (9), means adapted to measure the temperature of the liquid in the liquid outlet (11) and said means adapted to measure the flow value of the liquid.

13. Arrangement according to claim 2 characterized in that the arrangement comprises means (16) for measuring the impedance in the circuit comprising the needle (2) and the ground electrode (4) and means (18) adapted to measure the output power of the radio frequency generating device (10).

14. Arrangement according to claim 3 characterized in that the arrangement comprises means (16) for measuring the impedance in the circuit comprising the needle (2) and the ground electrode (4) and means (18) adapted to measure the output power of the radio frequency generating device (10).

15. Arrangement according to claim 3 characterized in that the arrangement comprises a control unit (26), said control unit is: adapted to receive values of:
a) the liquid flow in the channels (13a, 13b) of the needle (2),
b) the temperature as measured by the thermosensor(s) incorporated in the needle (2), and the temperature of the liquid in the liquid inlet (9),
c) the impedance in the circuit comprising the needle (2) and the ground electrode (4),
d) the output power of said radio frequency generating device (10) provided with means adapted to calculate the tissue temperature depending on the values of a) and b), and adapted to send signals (30) to the radio frequency generating device (10) with instructions to decrease or increase the output power of said radio frequency generating device depending on if the tissue temperature is intended to be decreased or increased.

16. Arrangement according to claim 3 characterized in that the arrangement comprises a control unit (26), said control unit is: adapted to receive values of:
a) the liquid flow in the channels (13a, 13b) of the needle (2) and
b) the temperature of the liquid in the liquid inlet (9) and the temperature of the liquid in the liquid outlet (11);
c) the impedance in the circuit comprising the needle (2) and the ground electrode (4),
d) the output power of said radio frequency generating device (10) provided with means adapted to calculate the tissue temperature depending on the values of a) and b), and adapted to send signals (30) to the radio frequency generating device (10) with instructions to decrease or increase the output power of said radio frequency generating device depending on if the tissue temperature is intended to be decreased or increased.
